(19) United States
(12) Patent Application Publication
(10) Pub. No.: US 2011/0046077 A1
(43) Pub. Date: Feb. 24, 2011

(54) PROCESS FOR THE EXTRACTION OF MANGIFERIN AND ISOMANGIFERIN

(75) Inventors: Pascale Talamond, Eyragues (FR); Laurence Mondolot, Montpellier (FR); Annick Gargadennec, Montpellier (FR); Alexandre De Kochiko, Montpellier (FR); Serge Hamon, Teyran (FR); Alain Fruchier, Montamaud (FR); Claudine Campa, Teyran (FR)

Correspondence Address: MARSHALL, GERSTEIN & BORUN LLP, 233 SOUTH WACKER DRIVE, 6300 WILLIS TOWER, CHICAGO, IL, 60606-6357, US

(73) Assignee: Institut De Recherche Pour Le Developpement (IRD), Marseille 2 (FR)

(21) Appl. No.: 12/936,859

(22) PCT Filed: Apr. 10, 2009
(86) PCT No.: PCT/EP09/54349

§ 371 Date: Oct. 7, 2010

(30) Foreign Application Priority Data
Apr. 11, 2008 (FR) 0852468

(51) Int. Cl. A61K 31/7048; C07H 1/08; A61K 8/60; A61P 3/10; A61P 39/06; A61P 37/08; A61Q 19/00

(52) U.S. Cl. 514/23; 536/128

(57) ABSTRACT

The present invention relates to methods for extracting and isolating glycosyl xanthone derivatives, in particular mangiferin and isomangiferin, from plants of the Rubiaceae family, especially of the Coffea genus. The invention also relates to extracts obtained using such methods, as well as compositions comprising such extracts that are useful in the cosmetic and pharmaceutical industry.

### RELATED APPLICATIONS

This application claims priority to French Patent Application No. FR 08 524 68 filed on Apr. 11, 2008, which is incorporated herein by reference in its entirety.

### FIELD OF THE INVENTION

The present invention relates to a process for obtaining C-glycosyl xanthone derivatives, in particular mangiferin and isomangiferin, from plants of the Rubiaceae family, such as plants of the Coffea genus.

### BACKGROUND OF THE INVENTION

Mangiferin and isomangiferin are natural products present in a number of plants. They are C-glycosyl xanthone derivatives that have numerous advantageous properties from the cosmetic and pharmaceutical viewpoint. In fact, it has been demonstrated that mangiferin, just like other xanthone derivatives, has antidiabetic (Miura et al., 2001), antioxidant (Garrido et al., 2004), antiallergic (Pinto et al., 2005), antihyperlipidaemic (Muruganandan et al., 2005) and anticarcinogenic (Pinto et al., 2005) properties as well as cardiotonic and diuretic properties (GB 1 099 764). It has also been suggested that mangiferin could be used in the treatment of diseases and clinical conditions caused by the herpes virus (GB 2 108 383). Furthermore, due to its anti-collagenase, anti-elastase, anti-tyrosinase and anti-radical activities and to its photoprotective activities in the region of ultraviolet (UV) radiation, mangiferin is of use in protecting the skin from UV radiation, in improving its structural quality and in helping to combat biological and/or radiation-induced skin ageing (WO 96/16632). Moreover, it has been demonstrated that mangiferin activates the expression of heat-shock proteins and inhibits the expression of matrix metalloproteases, thus improving the cellular response to heat shock (US 2006/0088560).

Mangiferin and isomangiferin (the chemical structures of which are presented on FIG. 1) belong to the xanthone family. This family forms a large group of natural products which are generally found only in some families of higher plants, in lichens and in fungi (Sultanbawa, 1980; Hostettmann and Hostetmann, 1989). An analysis of the scientific literature has shown that 515 different natural xanthones were identified from January 2000 to December 2004 (i.e., within only 5 years), 278 of these xanthones being new xanthones discovered for the first time (Viera and Kijjoa, 2005). Despite their high biochemical diversity, the xanthones of higher plants are mainly associated with the Clusiaceae and Gentianaceae families. They are occasionally found in phylogenetically distant families, such as the Iridaceae, Liliaceae, Anacardiaceae, Euphorbiaceae or Verbenaceae. Thus, mangiferin, which was initially isolated from Mangifera indica L. (Anacardiaceae), is naturally present in a number of species of the families of the Fabaceae, Gentianaceae, Anacardiaceae, Flacourtiaceae, Polypodiaceae, Guttiferae, Leguminosae, Hippocrateaceae, Sapotaceae, Convolvulaceae, Liliaceae, Iridaceae and Poaceae. Just like the other natural xanthones identified to date, neither mangiferin nor isomangiferin has been isolated from plants of the family of the Rubiaceae, to which in particular gardenia (genus Gardenia), cinchona (genus Cinchona) and the coffee plant (genus Coffea) belong.

Due to its impact on the quality of coffee, a great deal of information exists on the chemical composition of green or roasted coffee beans. The majority of studies have been carried out on cultivated species, such as Coffea arabica and Coffea canephora. The biochemical composition of coffee beans has also been studied for some of the 103 wild species identified to date (Anthony et al., 1993; Campa et al., 2005a; Campa et al., 2005b), revealing that sugars, lipids, chlorogenic acids, amino acids, caffeine and trigonelline are generally the main compounds which accumulate during the growth of the coffee bean (Tressl, 1989; Ho et al., 1993). The inter- and intra-species diversity of the metabolic content has been extensively studied (Clifford, 1985; Rogers, 1999) and the results obtained have shown that chlorogenic acids, which are soluble phenolic compounds, strongly accumulate in the green beans, except in the case of the wild species, such as Coffea pseudozanguebariae. In contrast, very few biochemical analyses have been carried out on the leaves of wild or cultivated species. The most recent studies have evaluated the caffeine and trigonelline contents in the leaves of C. arabica (Zheng and Ashihara, 2004) and the chlorogenic acid content in the leaves of C. pseudozanguebariae (Bertrand et al., 2003) and C. canephora (Mondolot et al., 2006).

### SUMMARY OF THE INVENTION

The inventors have, for the first time, demonstrated the presence of xanthone derivatives, in particular C-glycosyl xanthone derivatives, in plants of the family of the Rubiaceae and have developed methods for the extraction and isolation of such derivatives. In particular, the inventors have shown that the leaves of some species of coffee plants comprise significant amounts of C-glycosyl xanthones, in particular of mangiferin and isomangiferin. They have also shown that mangiferin is present in the leaves of the species of Rondeletia odorata.

Thus, the present invention generally relates to processes for obtaining C-glycosyl xanthones from one or more plants belonging to one or more species of the Rubiaceae family. The plant of the Rubiaceae family may belong to a subfamily selected from the group consisting of Rubioideae, Cinchonoideae, Ixoroideae, and Antirheoideae.

The plants of the Rubiaceae family may belong to a genus selected from the group consisting of Acranthera, Acrobotrys, Acunaeanthus, Adina, Adinauclea, Agathisanthemum, Aidia, Aidiopsis, Airosperma, Aitchisonia, Alberta, Aleisanthia, Alibertia, Allaeophania, Alleizettella, Allenanthus Alseis, Amaioua, Amaracarpus, Amphiasma, Amphidasya, Ancylanthos, Anomanthodia, Antherostele, Anthorrhiza, Anthospermum, Antirhea, Aoranthe, Aphaenandra, Aphanocarpus, Appunia, Arachnothryx, Arcytophyllum, Argocoffeopsis, Argostemma Ariadne, Asemnantha Asperugalium, Asperula Astiella, Atractocarpus, Atractogyne, Augusta, Aulacocalyx, Badusa, Balmea, Bancalus, Bathysa, Batopedina, Becheria, Belonophora, Benkara, Benzonia, Berghesia, Bertiera, Bikkia, Blandibractea, Blepharidium, Bobea, Boholia, Borojoa, Bothriospora, Botryarrhena, Bouvardia, Brachytome, Bradea, Brenania, Breonadia, Breonia, Burchellia, Burttdavya, Byrsophyllum, Calanda, Callipeltis, Calochone, Calycophyllum, Calycosia, Calycosiphonia, Canephora, Canthium, Capirona, Captaincookia, Carpacoce, Carphalea, Carterella, Casasia, Catesbaea, Catunaregam, Cephalanthus, Cephalodendron, Ceratopyxis, Ceriscoides, Ceuthocarpus, Chaetostachydium, Chalepophyllum, Chamaepentas, Chapelieria, Chassalia, Chazaliella, Chimarrhis, Chiococca, Chione, Chomelia, Choulettia, Cigarrilla, Cinchona, Cladoceras, Clarkella, Coccochondra, Coccocypselum, Codaria, Coddia, Coelopyrena, Coelospermum, Coffea, Coleactina, Colletoecema, Commitheca, Condaminea, Conostomium, Conotrichia, Coprosma, Coptophyllum, Coptosapelta, Corynanthe, Coryphothamnus, Cosmibuena, Cosmocalyx, Coupoui, Coussarea, Coutaportla, Coutarea, Cowiea, Craterispermum, Cremaspora Cremocarpon, Crobylanthe, Crocyllis, Crossopteryx, Crucianella, Cruciata, Cruckshanksia, Crusea, Cuatrecasasiodendron, Cubanola, Cuviera, Cyclophyllum, Damnacanthus, Danais, Deccania, Declieuxia, Dendrosipanea, Dentella, Deppea, Diacrodon, Dialypetalanthus, Dibrachionostylus, Dichilanthe, Dictyandra, Didymaea, Didymochlamys, Didymoecium, Didymopogon, Didymosalpinx, Diodia, Dioecrescis, Dioicodendron, Diplospora, Discospermum, Diyaminauclea, Dolichodelphys, Dolicholobium, Dolichometra, Doricera, Duidania, Dunnia, Duperrea, Duroia, Durringtonia, Ecpoma, Eizia, Elaeagia, Eleuthranthes, Emmenopterys, Emmeorhiza, Eosanthe, Eriosemopsis, Erithalis, Ernodea, Etericius, Euclinia, Exostema, Fadogia, Fadogiella, Fagerlindia, Faramea, Ferdinandusa, Feretia, Fergusonia, Fernelia, Flagenium, Flexanthera, Gaertnera, Galiasperula, Galiniera, Galium, Gallienia, Galopina, Gardenia, Gardeniopsis, Genipa, Gentingia, Geophila, Gilipus, Gillespiea, Gleasonia, Glionnetia, Glossostipula, Gomphocalyx, Gonzalagunia, Gouldia, Greenea, Greeniopsis, Guettarda, Gynochthodes, Gynopachis, Gyrostipula, Habroneuron Haldina, Hallea, Hamelia, Hayataella, Hedstromia, Hedyotis, Hedythyrsus, Heinsenia, Heinsia, Hekistocarpa, Henlea, Henriquezia, Heterophyllaea, Hillia, Himalrandia, Hindsia, Hintonia, Hippotis, Hitoa, Hodgkinsonia, Hoffmannia, Holstianthus, Homollea, Homolliella, Hondbessen, Houstonia, Hutchinsonia, Hydnophytum, Hydrophylax, Hymenocnemis, Hymenocoleus, Hymenodictyon, Hyperacanthus, Hypobathrum, Hyptianthera, Indopolysolenia, Isertia, Isidorea, Ixora, Jackiopsis, Janotia, Jaubertia, Javorkaea, Joosia, Jovetia, Kailarsenia, Kajewskiella, Keenania, Keetia, Kelloggia, Kerianthera, Khasiaclunea, Klossia, Knoxia, Kochummenia, Kohautia, Kraussia, Kutchubaea, Ladenbergia, Lagynias, Lamprothamnus, Lasianthus, Lathraeocarpa, Lecananthus, Lecariocalyx, Lelya, Lemyrea, Lepidostoma, Leptactina, Leptodermis, Leptomischus, Leptoscela, Leptostigma, Leptunis, Lerchea, Leroya, Leucocodon, Leucolophus, Limnosipanea, Lindenia, Litosanthes, Lucinaea, Luculia, Lucya, Ludekia, Macbrideina, Machaonia, Macrocnemum, Macrosphyra, Maguireocharis, Maguireothamnus, Malanea, Manettia, Manostachya, Mantalania, Margaritopsis, Maschalocorymbus, Maschalodesme, Massularia, Mastixiodendron, Mazaea, Melanopsidium, Menestoria, Mericarpaea, Merumea, Metadina, Meyna, Micrasepalum, Microphysa, Mitchella, Mitracarpus, Mitragyna, Mitrasacmopsis, Mitriostigma, Molopanthera, Monosalpinx, Montamans, Morelia, Morierina, Morinda, Morindopsis, Motleyia, Mouretia, Multidentia, Mussaenda, Mussaendopsis, Mycetia, Myonima, Myrioneuron, Myrmecodia, Myrmeconauclea, Myrmephytum, Nargedia, Nauclea, Neanotis, Neblinathamnus, Nematostylis, Nenax, Neobertiera, Neoblakea, Neobreonia, Neofranciella, Neogaillonia, Neohymenopogon, Neolamarckia, Neolaugeria, Neoleroya, Neonauclea, Neopentanisia, Nernstia, Nertera, Nesohedyotis, Neurocalyx, Nichallea, Nodocarpaea, Normandia, Nostolachma, Ochreinauclea, Octotropis, Oldenlandia, Oldenlandiopsis, Oligocodon, Omiltemia, Opercularia, Ophiorrhiza, Ophryococcus, Oregandra, Osa, Otiophora, Otocalyx, Otomeria, Ottoschmidtia, Oxyanthus, Oxyceros, Pachystigma, Pachystylus, Paederia, Pagamea, Pagameopsis, Palicourea, Pamplethantha, Paracephaelis, Parachimarrhis, Paracorynanthe, Paragenipa, Paraknoxia, Parapentas, Paratriaina, Pauridiantha, Pausinystalia, Pavetta, Payera, Pelagodendron, Pentagonia, Pentaloncha, Pentanisia, Pentanopsis, Pentas, Pentodon, Peponidium, Perakanthus, Perama, Peratanthe, Peripeplus, Pertusadina, Petitiocodon, Phellocalyx, Phialanthus, Phitopis, Phuopsis, Phyllacanthus, Phyllis, Phyllocrater, Phyllomelia, Phylohydrax, Picardaea, Pimentelia, Pinarophyllon, Pinckneya, Pittoniotis, Placocarpa, Placopoda, Platycarpum, Plectroniella, Pleiocarpidia, Pleiocoryne, Pleiocraterium, Plocama, Plocaniophyllon, Poecilocalyx, Pogonolobus, Pogonopus, Polysphaeria, Polyura, Pomax, Porterandia, Portlandia, Posoqueria, Pouchetia, Praravinia, Pravinaria, Preussiodora, Prismatomeris, Proscephaleium, Psathura, Pseudaidia, Pseudogaillonia, Pseudogardenia, Pseudohamelia, Pseudomantalania, Pseudomussaenda, Pseudonesohedyotis, Pseudopyxis, Pseudosabicea, Psilanthus, Psychotria, Psydrax, Psyllocarpus, Pteridocalyx, Pterogaillonia, Pubistylus, Putoria, Pygmaeothamnus, Pyragra, Pyrostria, Ramosmania, Randia, Raritebe, Ravnia, Readea, Relbunium, Remijia, Rennellia, Retiniphyllum, Rhachicallis, Rhadinopus, Rhaphidura, Rhipidantha, Rhopalobrachium, Richardia, Riqueuria, Robynsia, Rogiera, Roigella, Rondeletia, Rothmannia, Rubia, Rudgea, Rustia, Rutidea, Rytigynia, Sabicea, Sacosperma, Saldinia, Salzmannia, Saprosma, Sarcocephalus, Sarcopygme, Schachtia, Schismatoclada, Schizenterospermum, Schizocalyx, Schizocolea, Schizostigma, Schmidtottia, Schradera, Schumanniophyton, Schwendenera, Scolosanthus, Scyphiphora, Scyphochlamys, Scyphostachys, Sericanthe, Serissa, Shaferocharis, Sherardia, Sherbournia, Siderobombyx, Siemensia, Simira, Sinoadina, Sipanea, Sipaneopsis, Siphonandrium, Sommera, Spathichlamys, Spermacoce, Spermadictyon, Sphinctanthus, Spiradiclis, Squamellaria, Stachyarrhena, Stachyococcus, Staelia, Standleya, Steenisia, Stelechantha, Stephanococcus, Stevensia, Steyermarkia, Stichianthus, Stilpnophyllum, Stipularia, Stomandra, Streblosa, Streblosiopsis, Striolaria, Strumpfia, Stylosiphonia, Suberanthus, Sukunia, Sulitia, Synaptantha, Syringantha, Tamilnadia, Tammsia, Tapiphyllum, Tarenna, Tarennoidea, Temnocalyx, Temnopteryx, Tennantia, Thecorchus, Theligonum, Thogsennia, Thyridocalyx, Timonius, Tobagoa, Tocoyena, Tortuella, Trailliaedoxa, Tresanthera, Triainolepis, Tricalysia, Trichostachys, Trukia, Tsiangia, Ucriana, Uncaria, Urophyllum, Valantia, Vangueria, Vangueriella, Vangueriopsis, Versteegia, Villaria, Virectaria, Warszewiczia, Webera, Wendlandia, Wernhamia, Wiasemskya, Wittmackanthus, Xanthophytum, Xantonnea, Xantonneopsis, Yutajea, and Zuccarinia.

In certain preferred embodiments, the plant of the Rubiaceae family belongs to a genus selected from the group consisting of Coffea and Rondeletia. Most preferably, the plant of the Rubiaceae family belongs to the Coffea genus. The extraction, isolation and purification processes developed by the inventors make it possible, in particular, to obtain xanthone derivatives, such as mangiferin and isomangiferin, which have advantageous cosmetic and/or pharmaceutical properties.

In certain preferred embodiments, the extraction process of the present invention is carried out using the aerial parts of coffee plants, in particular the leaves. In this case, the process of the invention exhibits, among other advantages, that of being able to be carried out throughout the year, since virtually all coffee plants are evergreen trees.

The process according to the present invention is characterized in that it comprises an extraction step carried out on starting material which has been lyophilized and reduced to a fine powder beforehand. This extraction is carried out with a water/polar organic solvent mixture, preferably a water/alcohol mixture, more preferably still a water/methanol mixture, in a 20/80 ratio by volume. The extraction step is preferably carried out by sonication. The extraction produces an extract comprising at least one C-glycosyl xanthone, in particular mangiferin and/or isomangiferin.

The process according to the present invention can further comprise a step which makes it possible to isolate at least one C-glycosyl xanthone from the extract obtained above. This step can be carried out using any appropriate method, for example chromatography. According to the process of the invention, the extract is submitted to medium-pressure liquid chromatography on a cellulose column eluted first with water, in order to obtain a fraction 1 which comprises mangiferin, and then with a water/methanol mixture, in order to obtain a fraction 2 which comprises isomangiferin. Mangiferin can be obtained substantially pure by gel filtration of fraction 1. Medium-pressure liquid chromatography of fraction 2 on a cellulose column eluted with a water/ethanol mixture provides substantially pure isomangiferin.

The invention also relates to extracts comprising at least one C-glycosyl xanthone which are obtained from plants of the family of the Rubiaceae, such as coffee plants. In particular, the extracts can comprise mangiferin, isomangiferin or a mixture of the two. Preferably, the extracts are obtained using one of the processes described here or a variant of these processes. In some preferred embodiments, mangiferin or isomangiferin is the major component of an extract according to the invention.

The invention also relates to substantially pure C-glycosyl xanthones, in particular mangiferin and isomangiferin, obtained from plants of the family of the Rubiaceae, in particular from coffee plants. Preferably, the xanthones are obtained using one of the processes described here or a variant of these processes.

Finally, the invention also relates to pharmaceutical or cosmetic preparations comprising a substantially pure C-glycosyl xanthone or an extract comprising at least one C-glycosyl xanthone, in which preparations the C-glycosyl xanthone or the extract is obtained from plants of the family of the Rubiaceae, in particular from coffee plants. Preferably, the extract or the C-glycosyl xanthone is obtained using one of the extraction processes described here or a variant of these processes. A pharmaceutical or cosmetic preparation according to the invention can optionally comprise at least one additional active principle.

A more detailed description of some preferred embodiments of the invention is given below.

### BRIEF DESCRIPTION OF THE FIGURES

FIG. 1 shows the chemical structures of mangiferin (1) and isomangiferin (2).

FIG. 2 shows the absorption spectrum of mangiferin (and isomangiferin) recorded in 2 mM phosphoric acid in water and methanol (55:45, vol:vol).

### DETAILED DESCRIPTION OF THE INVENTION

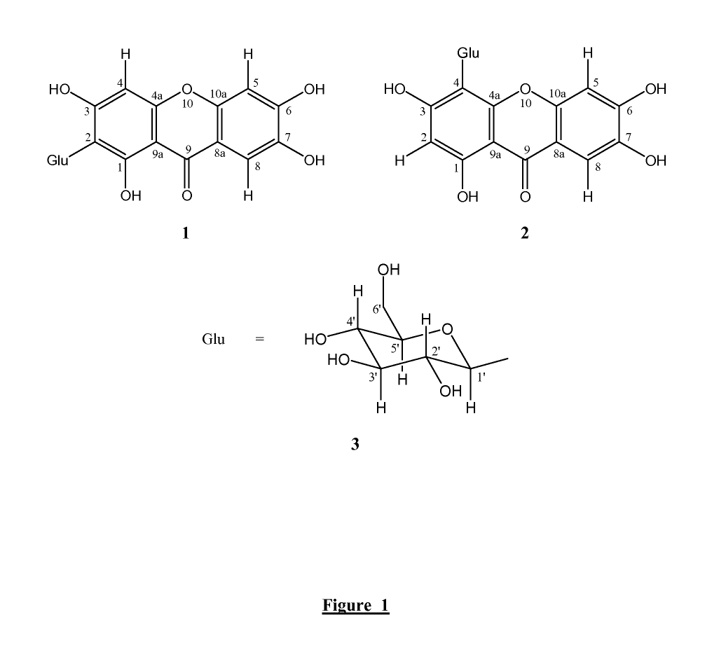

Generally, the present invention relates to processes for obtaining glycosyl xanthones, in particular C-glycosyl xanthones. In the context of the invention, the term “glycosyl xanthone” is understood to encompass any molecule having a xanthone nucleus (i.e., a tricyclic structure, also known as dibenzo-gamma-pyrone or 9-oxoxanthene) which has been submitted to a glycosylation, namely the attachment of a group corresponding to the formula 3 presented in FIG. 1. A xanthone is “C-glycosylated” when it carries a glucose molecule attached to one (or more) of its carbon atoms. In the case of mangiferin and isomangiferin, the xanthone nucleus carries four hydroxyl radicals substituted on the 1, 3, 6 and 7 carbons of the two phenol nuclei. The glycosylation is carried out on carbon 2 of the xanthone nucleus in the case of mangiferin and on carbon 4 in the case of isomangiferin.

The processes of the present invention are carried out starting from plants of the family of the Rubiaceae, in particular coffee plants (genus Coffea).

Coffee plants are of tropical African origin but are cultivated all over the world in tropical and subtropical regions. Coffee plants are perennial plants which come in the form of bushes or trees, with generations of approximately thirty years. Their leaves are lanceolate and are a dark and glossy green. Their fruits (commonly known as “cherries”) remain green for a long time and take several months to ripen. For the cultivated species, the fruits can be harvested when they begin to turn dark red.

Coffee plants are generally cultivated for their beans which, after roasting, give coffee, one of the most commonly consumed drinks in the world. In world trade, coffee is the second biggest export product in terms of value. The cultivation and the marketing thereof provide a livelihood to more than 125 million people in Latin America, Africa and Asia. Two species are cultivated in the intertropical region, Coffea arabica(approximately 70% of production) and Coffea canephora (approximately 30%). In addition to these two species, which are the most widely cultivated, botanists have described approximately one hundred wild species, which reflects a very high genetic diversity (Davis et al., 2006).

The coffee plants suitable for use in the process of the present invention can belong to any appropriate species of the genus Coffea. Thus, a coffee plant used in the process of the invention can be of a species generally cultivated for the production of coffee, or, alternatively, of a wild species (i.e. of a species which is not cultivated for the production of coffee). In some embodiments, the plants used in the process according to the invention are of the same species of coffee plant. Alternatively, the plants can originate from different species of coffee plant.

Generally, the coffee plants which can be used in the extraction process of the present invention can be chosen, for example, from Coffea abbayesii, Coffea abeokutae, Coffea affinis, Coffea alleizettii, Coffea ambanjensis, Coffea ambongensis, Coffea andrambovatensis, Coffea ankaranensis, Coffea anthonyi, Coffea arabica L., Coffea arenesiana, Coffea augagneurii, Coffea bakossii, Coffea benghalensis, Coffea bertrandii, Coffea betamponensis, Coffea bissetiae, Coffea boinensis, Coffea boiviniana, Coffea bonnieri, Coffea brevipes, Coffea bridsoniae, Coffea buxifolia, Coffea canephora, Coffea carrissoi, Coffea charrieriana, Coffea commersoniana, Coffea congensis, Coffea costatifructa, Coffea coursiana, Coffea dactylifera, Coffea decaryana, Coffea dewevrei, Coffea dubardii, Coffea eugenioides, Coffea fadenii, Coffea farafanganensis, Coffea fotsoana, Coffea fragilis, Coffea gallienii, Coffea grevei, Coffea heimii, Coffea heterocalyx, Coffea homollei, Coffea humbertii, Coffea humblotiana, Coffea humilis, Coffea jumellei, Coffea kapakata, Coffea khasiana, Coffea kianjavatensis, Coffea kihansiensis, Coffea kimbozensis, Coffea kivuensis, Coffea klainii, Coffea labatii, Coffea lancifolis, Coffea leonimontana, Coffea leroyi, Coffea liaudii, Coffea liberica, Coffea ligustroides, Coffea littoralis, Coffea lulandoensis, Coffea macrocarpa, Coffea magnistipula, Coffea mangoroensis, Coffea manombensis, Coffea mapiana, Coffea mauritiana, Coffea mayombensis, Coffea mcphersonii, Coffea millotii, Coffea minutiflora, Coffea mogenetii, Coffea mongensis, Coffea montekupensis, Coffea montis-sacri, Coffea moratii, Coffea mufindiensis, Coffea myrtifolia, Coffea perrieri, Coffea pervilleana, Coffea pocsii, Coffea pseudozanguebariae, Coffea pterocarpa, Coffea quillou, Coffea racemosa, Coffea rakotonasoloi, Coffea ratsimamangae, Coffea resinosa, Coffea rhamnifolia, Coffea richardii, Coffea rupestris, Coffea sahafaryensis, Coffea sakarahae, Coffea salvatrix, Coffea sambavensis, Coffea schliebenii, Coffea sessiliflora, Coffea sp Moloundou, Coffea stenophylla, Coffea tetragons, Coffea togoensis, Coffea travancorensis, Coffea tricalysioides, Coffea tsirananae, Coffea vatovavyensis, Coffea vavateninensis, Coffea vianneyi, Coffea vohemarensis, Coffea wightiana, Coffea zanguebariae, and hybrids thereof.

In some preferred embodiments, the coffee plants used in an extraction process of the invention are chosen from Coffea arabica, Coffea eugenioides, Coffea heterocalyx, Coffea pseudozanguebariae, Coffea sp Moloundou, and hybrids thereof. Coffea sp Moloundou is now called Coffea anthonyi.

The process of the present invention is generally carried out starting from the whole or a portion of the aerial part of plants of the family of the Rubiaceae, in particular coffee plants. In the context of the present invention, the term “aerial part of a plant” is understood to mean the portion of the plant which is commonly called foliage and which is found above the ground. Generally, the aerial part or foliage of a plant comprises the leaves, stems, flowers and fruits. In certain preferred embodiments, the extraction process of the invention is carried out using coffee plant leaves. As mentioned above, coffee plant leaves are generally persistent and thus constitute a virtually permanent source of raw material.

The inventors have shown that C-glycosyl xanthones, in particular mangiferin, are present at a higher concentration in young leaves than in older leaves of Coffea pseudozanguebariae (see Examples). Thus, in certain embodiments, an extraction process according to the invention is preferably carried out with young coffee plant leaves. In the context of the present invention, the term “young leaves” is understood to mean leaves having a length which is at most half the length of the adult leaf. It is highly probable that the change in the concentration of C-glycosyl xanthone as a function of the stage of development of the leaves will vary from one species to another. A person skilled in the art will know how to quantify the presence of C-glycosyl xanthones in coffee plant leaves, to study their variations as a function of the development of the leaves and to determine the stage of development corresponding to the highest concentration. For example, such a determination can be carried out by an HPLC analysis method, such as that developed by the inventors (see Examples).

In what follows, the description of the invention is given mainly with reference to the use of leaves of coffee plants. It is understood that the invention is not limited to this specific case and that the use of other plants of the Rubiaceae family and/or of other portions of the aerial part of these plants is encompassed within the present invention.

A person skilled in the art will understand that many extraction and isolation methods can be used in order to obtain at least one C-glycosyl xanthone from coffee plant leaves, the nature of the extraction method not being a critical or limiting element.

The inventors have developed a specific process, characterized in that it consists in employing a step of extraction of coffee plant leaves with a mixture of water and of polar organic solvent in order to obtain an extract comprising at least one C-glycosyl xanthone.

Before extraction, the coffee plant leaves are ground, preferably in a powder form, for example in a fine powder. The grinding can be carried out at ambient temperature or under cold conditions by means of any appropriate method (for example using a pestle and mortar system). The leaves are preferably dehydrated beforehand by lyophilization. Alternatively, (and generally with a lower yield), the grinding can be carried out on fresh leaves (that is to say, leaves that have not been dehydrated by lyophilization). In this latter embodiment, the leaves are frozen before being ground.

According to the present invention, after grinding, the leaves are extracted with a mixture of water and of polar organic solvent. The polar organic solvent is advantageously chosen from linear or branched C₁-C₃ alcohols and mixtures of these alcohols in appropriate proportions. In certain embodiments, the polar organic solvent is an alcohol, such as methanol, ethanol or a mixture of methanol and ethanol. In a preferred embodiment, the mixture of water and of polar organic solvent is a mixture of water and methanol. Preferably, such a mixture comprises less water than methanol. For example, the methanol and water are present in a volume ratio ranging from approximately 65/35 to approximately 90/10, preferably approximately 80/20.

Extraction can be carried out by any suitable method, in particular any method which promotes rupture of the plant cells and/or subcellular membranes of plant cells. These methods can be based on mechanical, chemical and/or biochemical techniques. Such methods are known in the art and include, for example, mechanical grinding (using, for example, pestle and mortar, grinder of Potter-Elvehjem type, or grinder of Dounce type), mechanical shredding (for example, Waring Blender™, or Virtis grinder), sonication, cavitation, osmotic shock, use of compounds which promote homogenization (detergents, abrasive agents, and the like), use of lytic enzymes (proteases, nucleases, lipases), and the like. Any appropriate combination of these methods can also be used in the extraction process of the present invention.

In some embodiments, the extraction is carried out by sonication. A person skilled in the art will know how to determine the conditions and duration of the sonication step in order to successfully conclude the extraction and will also know how to adapt these conditions and this duration in order to optimize the extraction. The factors which may be taken into account for such a step include, without limitation, the amount of starting material (i.e., the ground leaves), the nature of the water/polar organic solvent mixture used, the proportion of amount of starting material to volume of the water/organic solvent mixture used, and the like. Sonication can be carried out at ambient temperature or at low temperature. As sonication produces heat, it may be preferable to carry out this stage under cold conditions (for example, “on ice” and in a cold chamber, i.e., at around 0-5° C.).

The extraction step (i.e., the extraction with a water/polar organic solvent mixture, accompanied or not accompanied by sonication) can be repeated several times.

The extraction provides an extract which comprises, among other compounds, at least one C-glycosyl xanthone. In certain preferred embodiments, an extract obtained from coffee plant leaves as described here comprises at least mangiferin, isomangiferin or a mixture of the two. In the context of the present invention, the term “extract” is understood to mean any substance obtained by a physical, chemical and/or biotechnological operation starting from coffee plant leaves and/or from cells of coffee plant leaves. Preferably, with respect to the raw material (dried coffee plant leaves), an extract is enriched in C-glycosyl xanthone(s) (i.e., it comprises a higher content of C-glycosyl xanthone(s) than the dried leaves).

In some embodiments, the extract obtained from coffee plant leaves is the final product of the process of the invention. Such an extract can be in the liquid form or in the form of a powder after drying by atomization, evaporation and/or lyophilization. In other embodiments, the process of the invention further comprises a step which makes it possible to isolate at least one C-glycosyl xanthone from an extract obtained from coffee plant leaves.

Starting from an extract as described above, a person skilled in the art can develop a great variety of methods for isolating the xanthone derivative(s) present in the extract.

The method developed by the inventors comprises using liquid chromatography, more accurately medium-pressure liquid chromatography. More specifically, according to the process of the invention, an extract obtained from coffee plant leaves is submitted to medium-pressure liquid chromatography on a cellulose column. Elution of this column with water provides a first fraction (fraction 1) which comprises mangiferin. After elution of fraction 1, a second elution of the cellulose column with a mixture of water and alcohol (for example, water/methanol in a 10/90 ratio by volume) provides a second fraction (fraction 2) which comprises isomangiferin.

The mangiferin present in fraction 1 can be obtained substantially pure, for example, by subjecting fraction 1 to gel filtration chromatography, in particular on a column of Sephadex® LH20 beads (i.e., a dextran derivative composed of glucose chains bonded via glycosidic bonds). Elution of this column with water provides substantially pure mangiferin. In the context of the present invention, when the term “substantially pure” is used to characterize a C-glycosyl xanthone, it relates to a C-glycosyl xanthone having a purity of at least approximately 90%, preferably of at least approximately 95%, more preferably still of at least approximately 97%, for example 98%, 99% or more. If desired, the mangiferin thus obtained can be crystallized (for example by lyophilization). The dry purified mangiferin exists in the form of prismatic needles which are pale yellow in colour.

The isomangiferin present in fraction 2 can be obtained substantially pure by subjecting fraction 2 to medium-pressure liquid chromatography, preferably on a cellulose column eluted with an alcohol/water mixture (for example, an ethanol/water mixture in an 80/20 ratio by volume). If desired, the isomangiferin thus obtained can be crystallized (for example by lyophilization). The dry purified isomangiferin exists in the form of prismatic needles which are pale yellow in color.

The extracts obtained from coffee plant leaves and which comprise at least one C-glycosyl xanthone are covered by the present invention. In such extracts, the C-glycosyl xanthone (for example mangiferin or isomangiferin) can be present at any concentration.

The invention also relates to the substantially pure C-glycosyl xanthones extracted and isolated from coffee plant leaves.

Thus, in a preferred embodiment, the present invention provides extracts comprising a mixture of mangiferin and isomangiferin. In another preferred embodiment, the present invention provides mangiferin which is substantially pure or present in an extract. In yet another preferred embodiment, the present invention provides isomangiferin which is substantially pure or present in an extract. Preferably, the extracts and the C-glycosyl xanthones are obtained according to one of the processes described herein or a variant of these processes.

The invention also relates to pharmaceutical, parapharmaceutical or cosmetic preparations comprising a substantially pure C-glycosyl xanthone or comprising an extract containing at least one C-glycosyl xanthone as defined above. Preferably, the C-glycosyl xanthone is mangiferin or isomangiferin.

Due to the properties of mangiferin (and of some of its derivatives) mentioned above, a cosmetic composition according to the invention can be used to limit the harmful effects of UV radiation on the skin, lips and hair, to improve the structural quality of the skin, to combat ageing of the skin and/or to prevent or reduce the effects of temperature variation on the skin, lips and hair.

A cosmetic composition according to the invention can be used as is or alternatively can be incorporated in a body care or cosmetic product. Thus, a cosmetic composition of the invention can be added to creams or lotions for the face, hands, feet or body (for example, day creams, night creams, body milks, detergents and soaps, lotions, milks, gels or foams for caring for the skin); makeup products; self-tanning creams, gels, oils or lotions; sunscreens; hair products (for example, shampoos, conditioners, coloring products, styling creams, gels or foams); shaving and aftershave products; lip balms; and the like.

A cosmetic composition of the present invention can be formulated in a solid, semisolid or liquid form. The choice of the formulation will generally be made according to the application for which the composition is intended. Formulations suitable for cosmetic use are known in the art and include, for example, simple emulsions (for example, oil-in-water or water-in-oil emulsions), multiple emulsions, microemulsions, aqueous or aqueous/alcoholic gels, oils, aqueous solutions or aqueous/alcoholic solutions, foams, creams, milks, lotions, pastes, sticks, powders, pencils, and the like.

For the preparation of such formulations, an extract or a C-glycosyl xanthone of the invention can be mixed with at least one appropriate excipient (for example, vegetable or mineral oils, vegetable or mineral waxes, silicones, alcohols, fatty acids, lanolin, water, and the like) or can be incorporated in vectors of liposome, macrosphere, microsphere, nanosphere, macroparticle, microparticle, nanoparticle, macrocapsule, microcapsule or nanocapsule type or also can be absorbed on powdery organic polymers, talcs, bentonites and other inorganic carriers.

A cosmetic composition of the invention can also comprise additives, such as antibacterial adjuvants, fragrances, extracted and/or synthetic lipids, gellifying and viscosifying polymers, surfactants, emulsifiers, and the like.

Generally, a cosmetic composition of the invention comprises an effective amount of an extract or C-glycosyl xanthone, that is to say an amount of an extract or of C-glycosyl xanthone that is sufficient to play its intended role or perform its designated action (for example, the intended role or designated action may be to provide effective photoprotection from UV radiation). For example, in some embodiments, a cosmetic composition of the invention can comprise between approximately 0.01% and approximately 5% by weight of extract or of C-glycosyl xanthone in the powder form or between approximately 0.01% and approximately 25% by weight of extract or of C-glycosyl xanthone in the encapsulated form.

The compositions for cosmetic use of the present invention can further comprise at least one additional cosmetic active principle (i.e., in addition to the extract or the C-glycosyl xanthone). The term “cosmetic active principle” is understood to mean any compound or substance which can be used in caring for the body, skin, hair, and the like, and is generally applied locally. The cosmetic active principles which can be used in the present invention can belong to various families of compounds and substances, including plant extracts, marine extracts, tissue extracts, small synthetic molecules, and the like. Such active principles are known in the art. For example, an appropriate cosmetic active principle can advantageously be selected from substances which increase skin protection (for example, vitamins, ceramides, substances for combating free radicals, UV screening agents), substances which can have a healing effect on the skin (for example, proteins, hyaluronic acid, amino acids) or an anti-inflammatory effect, substances which limit the harmful effects of the sun (sunscreens), tanning and self-tanning products, substances which facilitate the good condition of the scalp and that of the hair (for example, minerals, vitamins, ceramides, protein extracts, mucopolysaccharides, flower and/or fruit acids), substances for combating ageing and/or wrinkles, toning products, detergents, substances having an activity with regard to skin sensitivity, and the like. In such cosmetic compositions of the invention, each additional active principle is generally present in an amount sufficient to exert its activity.

It is understood that a cosmetic composition of the present invention can also be incorporated in a preparation intended for the treatment of certain allergies, itching, irritation or red blotches of the skin, including the lips and scalp.

An extract or a C-glycosyl xanthone according to the invention can be administered as is or in the form of a pharmaceutical preparation or composition in the presence of at least one physiologically acceptable vehicle or excipient. In the context of the present invention, the term “physiologically acceptable vehicle or excipient” is understood to mean any medium or additive which does not interfere with the effectiveness of the biological activity of the active principle (in this instance, the extract of the C-glycosyl xanthone) and which is not excessively toxic to the patient, at the concentrations at which it is administered.

The pharmaceutical compositions of the present invention can be administered using any combination of dosage and administration route which is effective in producing the desired therapeutic effect. The exact amount to be administered can vary from one patient to another as a function of the age and the general condition of the patient, the nature and the seriousness of the disease, and the like. The administration route (oral, parenteral, rectal, pulmonary, nasal, cutaneous, transdermal, mucosal, and the like) can be chosen according to the nature of the disease and the desired therapeutic effect (for example, antidiabetic, antiallergic, antihyperlipidaemic, cardiotonic or diuretic effect of the extract or C-glycosyl xanthone of the invention). Administration can be local or systemic.

Formulation of a pharmaceutical composition of the present invention can vary according to the administration route and the dosage. After formulation with at least one physiologically acceptable vehicle or excipient, a pharmaceutical composition of the invention can be in any form appropriate for administration to a mammal, including man, for example in the form of tablets, including compressed tablets, sugar-coated pills, capsules, syrups, ointments, injectable solutions, suppositories, and the like. The person skilled in the art knows how to select the vehicles and excipients most appropriate for the preparation of a certain type of formulation. Thus, for example, excipients such as water, 2,3-butanediol, Ringer's solution, isotonic sodium chloride solution, synthetic mono- or diglycerides and oleic acid are often used for the formulation of injectable preparations. Liquid compositions, including emulsions, microemulsions, solutions, suspensions, syrups, elixirs, and the like, can be formulated in the presence of solvents, solubilizing agents, emulsifiers, oils, fatty acids and other additives, such as suspending agents, preservatives, sweeteners, flavorings, viscosifying agents, colorants, and the like. Solid compositions for administration via the oral route can be formulated in the presence of an inert excipient, such as sodium citrate, and optionally of additives, such as binders, humectants, disintegrating agents, absorption accelerators, lubricating agents, and the like.

In certain embodiments, a pharmaceutical composition of the present invention is formulated for immediate release of the active principle (in this instance, a C-glycosyl xanthone, such as mangiferin or isomangiferin). Alternatively, a pharmaceutical composition can be formulated for prolonged release of the active principle. Numerous strategies are known in the art for bringing about prolonged release of an active principle, such as, for example, by increasing the residence time in the stomach, using coatings sensitive to the pH and/or to enzymatic actions, or bioadhesive coatings which cling to the walls of the stomach or intestines, or also using systems for encapsulation as mentioned above.

The pharmaceutical compositions of the present invention can further comprise at least one additional pharmaceutical active principle (i.e., in addition to the extract or the C-glycosyl xanthone). The term “pharmaceutical active principle” is understood to mean any compound or substance, the administration of which has a therapeutic effect or a beneficial effect on the health or general condition of a patient to which it is administered. Thus, a pharmaceutical active principle may be active against the disease which it is desired to treat by administration of the pharmaceutical composition; it may be active against a condition associated with the disease which it is desired to treat by administration of the pharmaceutical composition; or it may increase the availability and/or the activity of the C-glycosyl xanthone included in the pharmaceutical composition.

Examples of pharmaceutical active principles which can be present in a composition of the present invention include, without limitation, anticancer agents, anti-inflammatories, antihypertensive agents (for example, diuretics, beta-adrenergic blocking agents, calcium blockers, alpha-adrenoceptor agonists, sympatholytics and vasodilators), antipyretics, antipruritics and/or antihistamines, antidiabetics, hypo lipidaemic agents, antiarrhythmics, and the like.

The present invention also relates to a treatment method comprising a step in which an effective amount of a pharmaceutical composition described herein is administered to a patient. In particular, this method can be used for the treatment of a disease or clinical condition for which the administration of a C-glycosyl xanthone having antidiabetic, antioxidant, antiallergic, antihyperlipidaemic, anticarcinogenic, cardiotonic and/or diuretic properties is beneficial. In the context of the present invention, the term “treatment” is understood to mean a method having the aim (1) of slowing down or preventing the onset of a disease or clinical condition; (2) of slowing down or halting the progression of, the worsening of or the deterioration in the symptoms of the disease; (3) of improving the symptoms of the disease; and/or (4) of curing the disease. A treatment can be administered before the onset of the disease, for a preventive action, or it can be administered after initiation of the disease, for a therapeutic action. A patient is generally a mammal, preferably a human.

Unless otherwise stated, all the technical and scientific terms used here have the same meanings as those commonly understood by an ordinary expert in the field to which this invention belongs. All the publications, patent applications, patents and other references mentioned here are incorporated by reference.

The following examples and figures are presented in order to illustrate some embodiments of the procedures described above and should under no circumstances be regarded as a limit on the scope of the invention.

### EXAMPLES

### Example 1

### Coffee Plant Leaves

Coffea pseudozanguebariae leaves were collected from trees cultivated in tropical greenhouses (natural light, temperatures of 25° C. at night and 28° C. during the day, and a relative humidity of 78-82%) at the IRD research center in Montpellier (France). Young leaves (less than 4 cm in length) were harvested from 5 different genotypes for the procedures for extraction, isolation and purification of the compounds. Four hundred (400) grams of collected leaves were immediately frozen in liquid nitrogen before lyophilization (72 hours). For the biochemical evaluation of the contents of the leaves, 3 axes of 5 nodes (formed by two opposing leaves) were selected from two trees aged 15 years. The nodes were classified from Node 1, for the youngest (juvenile leaves), to Node 5, for the oldest (adult leaves). Developing buds were not considered. Node 5 corresponds to leaves at the base of a new shoot on the lignified part of a branch. For each tree, leaves from the same Node were combined, weighed and immediately frozen in liquid nitrogen before being lyophilized.

### Example 2

### Extraction of Xanthone Derivatives

Coffea pseudozanguebariae leaves (80 g), lyophilized and ground to a powder, were extracted 3 times by sonication (20 minutes, 24 kHz, R.E.U.S.-GEX 180, Contes, France) with a mixture of methanol and water (MeOH:H₂O 8:2) at ambient temperature (3×700 ml). Methanol was subsequently removed by concentration. After lyophilization, the aqueous extract was subjected to medium-pressure liquid chromatography on a column (400 mm×47 mm, Büchi, Flawil, Switzerland) of microcrystalline cellulose (Avicel, Merck, Darmstadt, Germany) eluted with water, in order to obtain a fraction 1, and then with a mixture of methanol and water (MeOH:H₂O 9:1), in order to obtain a fraction 2. Fraction 1 was subsequently purified on a Sephadex LH20 column (500 mm×25 mm, Fluka, Basle, Switzerland) and diluted with water in order to obtain compound 1. In order to obtain compound 2, fraction 2 was subjected to medium-pressure liquid chromatography on a column (210 mm×47 mm, Büchi) of microcrystalline cellulose (Avicel, Merck) and eluted with a mixture of ethanol and water (EtOH:H₂O 8:2).

### Example 3

### Mass and Nuclear Magnetic Resonance (NMR) Spectrometry

The mass spectrometry analyses of compounds 1 and 2 were carried out with a Micromass Q-TOF spectrometer (Waters, Milford, Mass., United States) using a positive mode electrospray ionization source.

The NMR spectra of compounds 1 and 2 were recorded on an Avance DRX-400 spectrometer (Bruker-Biospin GmbH, Germany) at 400.13 MHz for ¹H and at 100.62 MHz for ¹³C. The chemical shifts are given in ppm/TMS with the ¹³C signal of d₆-DMSO at 39.98 ppm. The NMR spectra were interpreted using the gradient versions of the conventional COSY, HMQC and HMBC sequences.

### Example 4

### Identification of Compounds 1 and 2

The mass spectra obtained for compounds 1 and 2 suggest that these compounds are closely related isomers. The two spectra recorded exhibit a signal (M+H) at m/z 423. The ¹H spectrum recorded for 1 in d₆-DMSO exhibits three singlets at 7.371, 6.845 and 6.374 ppm and a 7-spin complex system between 3.0 and 5.0 ppm. The analysis of this second-order system revealed coupling constants typical of a glucose entity (see, for example, Silva and Pinto, Curr. Med. Chem., 2005, 12: 2481-2497): 4.594 (J=9.9 Hz, H-1′), 4.047 (J=9.9 and 8.4 Hz, H-2′), 3.202 (J=8.4 and 8.6 Hz, H-3′), 3.123 (J=8.6 and 9.2 Hz, H-4′), 3.171 (J=9.2, 5.9 and 1.8 Hz, H-5′), 3.688 (J=11.8 and 1.8 Hz, H-6′a) and 3.406 ppm (J=11.8 and 5.9 Hz, H-6′b). The chemical shift of C-1′ at 73.6 ppm suggests a C—C bond between the sugar and the aglycone. The other chemical shifts (Table 1) allowed it to be postulated that compound 1 is mangiferin (FIG. 1). This was confirmed by comparison with the spectra reported in the literature (Fujita and Inoue, 1982; Catalano et al., 1996) and with the spectra of an authentic sample of mangiferin.

TABLE 1
Chemical shifts recorded for compound 1 and compound 2 and
comparison with published data.

| | Mangiferin | | Isomangiferin | |
|---|---|---|---|---|
| | Fujita and | | Fujita and | |
| | Inoue | Compound 1 | Inoue | Compound 2 |
| C-1 | 161.6 | 162.3 | 161.5 | 161.8 |
| C-2 | 107.3 | 108.1 | 97.4 | 97.6* (6.23) |
| C-3 | 163.6 | 164.3 | 163.2 | 163.7 |
| C-4 | 93.3 | 93.8 (6.37) | 103.8 | 104.5* |
| C-4a | 156.1 | 156.7 | 155.7 | 156.5* |
| C-10a | 150.7 | 151.4 | 150.7 | 151.4 |
| C-5 | 102.5 | 103.0 (6.84) | 102.6 | 103.0 (6.83) |
| C-6 | 153.6 | 155.0 | 153.7 | 155.0* |
| C-7 | 143.7 | 144.4 | 143.6 | 144.3 |
| C-8 | 108.1 | 108.3 (7.37) | 107.9 | 107.9 (7.36) |
| C-8a | 111.7 | 112.0 | 111.4 | 111.7 |
| C-9 | 179.0 | 179.5 | 179.1 | 179.6 |
| C-9a | 101.2 | 101.8 | 101.7 | 102.2* |
| C-1′ | 73.0 | 73.6 | 73.2 | 73.8* |
| C-2′ | 70.3 | 70.7 | 70.5 | 71.2* |
| C-3′ | 78.8 | 79.5 | 78.6 | 79.1 |
| C-4′ | 70.5 | 71.1 | 70.9 | 71.4 |
| C-5′ | 81.3 | 82.1 | 81.1 | 81.9 |
| C-6′ | 61.4 | 62.0 | 61.4 | 62.1* |

*These signals are broad.

The ¹H spectrum of compound 2 in the same solvent also exhibits 3 singlets at 7.365, 6.829 and 6.229 ppm and is very similar to the spectrum of compound 1, except that the signals which can be assigned to the sugar entity are broad and did not allow the coupling constants to be determined. However, the ¹³C signals are those of the glucose entity as for compound 1. Comparison with the values published for 1,3,6,7-tetrahydroxanthone itself (Fraga and Chauduri, 1979) showed that, in compound 2, the glucose entity is attached to the 4 position of the xanthone. The ¹³C chemical shifts recorded and listed in Table 1, compared with those found in the literature, confirm that compound 2 is isomangiferin (FIG. 1).

### Example 5

### Biochemical Evaluation by HPLC Analysis

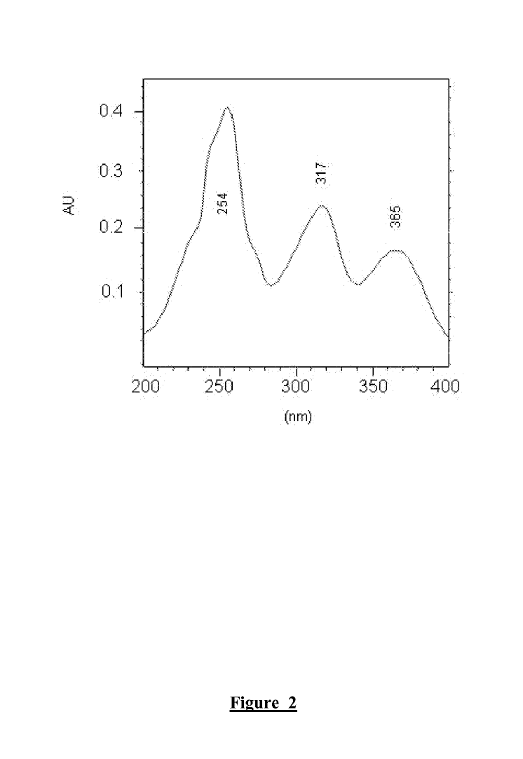

In order to quantify the xanthones in the leaf extracts, the leaves were ground to a fine powder and the phenolic compounds were extracted 3 times according to the method described by Ky et al., 2001. The xanthones and derivatives were identified from their retention times and UV absorption spectra (FIG. 2), using the HPLC analysis procedure described below.

The HPLC system used is equipped with a LiChrospher 100 RP-18 (5 μm) column (250 mm×4 mm, Merck, Darmstadt, Germany), a C18 guard column and a photodiode detector (Shimadzu, SPD-M20A). The elution system used (0.8 mL/min) comprises an eluent A, composed of an aqueous phosphoric acid solution (2M), and an eluent B, composed of methanol. The gradient used is as follows: 0 minute, 25% eluent B; 0-40 minutes, 80% eluent B, linear. The retention time and the spectral characteristics of each sample were compared with those of a reference sample of mangiferin (Extrasynthese, Lyons, France).

### Example 6

### Quantification of Compounds 1 and 2 in Coffee Plant Leaves

Compounds 1 and 2 were quantified in Coffea pseudozanguebariae leaves by HPLC analysis as described above. The results obtained are presented in Table 2 below. Mangiferin appears as the most abundant xanthone, with a percentage of more than 6% of the dry weight of the young leaves. This mangiferin content is higher than that determined in Mangifera zeylanica (Herath et al., 1970) or Cyclopia genistoides (Joubert et al., 2006). The mangiferin content in older leaves is lower and decreases during the growth of the leaves. In contrast, the isomangiferin content is constant during the development of the leaves and is generally lower than the mangiferin content.

TABLE 2
Content in mangiferin and isomangiferin as a
function of node.

Content (% dry weight)

| Node N^{o} | Mangiferin | Isomangiferin |
|---|---|---|
| 1 | 6.12 ± 0.66 | 0.29 ± 0.05 |
| 2 | 5.15 ± 0.68 | 0.29 ± 0.12 |
| 3 | 4.49 ± 0.93 | 0.27 ± 0.02 |
| 4 | 4.45 ± 0.49 | 0.28 ± 0.01 |
| 5 | 4.19 ± 1.54 | 0.22 ± 0.06 |

### Example 6

### Rondeletia Plant Leaves

Leaves from some Rubiaceae including Rondeletia odora were collected at the Montpellier Botanial Garden (Montpellier, France). The presence of mangiferin was shown by histolocalization in Rondeletia odora to leaves.

### REFERENCES

Anthony F. et al., “Biochemical diversity in the genus Coffea L.: chlorogenic acids, caffeine, and mozambioside contents”, Genet. Resour. Crop Evol., 1993, 40: 61-70.
Aritomi M. et al., “A new xanthone C-glucoside, position isomer of mangiferin, from Anemarrhena asphodeloides Bunge”, Chem. Pharm. Bull., 1970, 18: 2327-2333.
Bertrand C. et al., “Chlorogenic acid content swap during fruit maturation in Coffea pseudozanguebariae. Qualitative comparison with leaves”, Plant Sci., 2003, 165: 1355-1361.
Campa C. et al., “Diversity in bean caffeine content among wild Coffea species: evidence of a discontinuous distribution”, J. Food Chem., 2005a, 91: 633-637.
Campa C. et al., “Qualitative relationship between caffeine and chlorogenic acid contents among wild Coffea species”, J. Food Chem., 2005b, 93: 135-139.
Catalano S. et al., “A xanthone from Senecio mikanioides leaves”, Phytochemistry, 1996, 42: 1605-1607.
Clifford M. N., “Chemical and physical aspects of green coffee and coffee products”, In: Coffee: Botany, Biochemistry and Production of beans and beverage, 1985, Clifford M. N. & Wilson K. C. (ed.), Croom Helm: London, 305-375.
Davis A. et al., “An annotated taxonomic conspectus of the genus Coffea (Rubiaceae)”, Botanical Journal of the Linnean Society, 2006, 152(4): 465-512
Fujita M. et al., “Studies on the constituents of Iris florentina L. II.—C-Glucosides of xanthones and flavones from the leaves”, Chem. Pharm. Bull., 1982, 30: 2342-2348.
Fraga A. W. et al., “¹³C NMR spectroscopy of substituted xanthones. II.—¹³C NMR spectral study of polyhydroxy xanthones”, Tetrahedron, 1979, 35: 2035-2038.
Garcia D. et al., “Anthelminthic and antiallergic activities of Mangifera indica L. stem bark components vimang and mangiferin”, Phytother. Res., 2003, 17: 1203-1208.
Gorter K., “The precursor of Indian-yellow”, Bull. Jard. Hot. Buitenzorg, 1922, 4: 260-267.
Ho C. T. et al., “An overview of the Maillard reactions related to aroma generation in coffee”, In 15^{th} ASIC meeting proceedings, Montpellier (France), 1993, 519-527.
Herath P. et al., “Isolation of mangiferin from the bark of Mangifera zeylanica”, Phytochemistry, 1970, 9: 1141.
Hostettmann K. et al., “Xanthones”, In Methods in plant Biochemistry, Plant Phenolics, Harborne J. B. (ed.), Academic Press: London, 1989, 493-508.
Iseda S., “Mangiferin, the coloring matter of Mangifera indica. IV—Isolation of 1,3,6,7-tetrahydroxanthone and the skeletal structure of mangiferin”, Bull. Chem. Soc. of Jpn., 1957, 30: 625-629.
Jensen S. R. et al., “Chemotaxonomy and pharmacology of Gentianaceae”, In Gentianaceae—Systematics and Natural History, Struwe L and Albert V (ed.), Cambridge University Press: Cambridge, 2002, 573-631.
Joubert E. et al., “Use of NIRS for quantification of Mangiferin and Hesperidin contents of dried green Honeybush (Cyclopia genistoides) plant material”, J. Agric. Food Chem., 2006, 54: 5279-5283.
Ky L. et al., “Caffeine, trigonelline, chlorogenic acids and sucrose diversity in wild Coffea arabica L. and C. canephora P. accessions”, Food Chem., 2001, 75: 223-230.
Mondolot L. et al., “Evolution in caffeoylquinic acid content and histolocalization during leaf Coffea canephora development”, Ann. Bot., 2006, 98: 33-40.
Muruganandan S. et al., “Effect of mangiferin on hyperglycemia and atherogenicity in streptozotocin diabetic rats”, J. Ethnopharmacol., 2005, 97: 497-501.
Pinto M. M. M. et al., “Xanthones derivatives: new insights in biological activities”, Curr. med. chem., 2005, 12: 2517-2538.
Richardson P. M., “The taxonomic significance of C-glycosylxanthones in flowering plants”, Biochem. Syst. Ecol., 1983, 11: 371-375.
Richardson P. M. et al., “Mangiferin and isomangiferin in Acystopteris, Cystopteris, Gymnocarpium, and Woodsia”, J. Nat. Prod., 1983, 46: 747-749.
Rogers W. J. et al., “Changes to the content of sugars, sugar alcohols, myo-inositol, carboxylic acids and inorganic anions in developing grains from different varieties of Robusta (Coffea canephora) and Arabica (C. arabica) coffees”, Plant Sci., 1999, 149: 115-123.
Silva A. M. S. et al., “Structure elucidation of xanthone derivatives: Studies of NMR spectroscopy”, Curr. Med. Chem., 2005, 12: 2481-2497.
Sultanbawa M. U. S., “Xanthonoids of tropical plants”, Tetrahedron, 1980, 36: 1465-1506.
Tressl R., “Thermal generation of aromas”, In Formation of flavour components in roasted coffee, Parliament TH, McGorrin R J and Ho C, (ed.), American Chemical Society: Washington D.C.; 1989, 285-301.
Vieira M. M. et al., “Naturally-occurring Xanthones: recent developments”, Curr. Med. Chem., 2005, 12: 2413-2446.
Williams C. A. et al., “Flavonoid and xanthone patterns in bearded Iris species and the pathway of chemical evolution in the genus”, Biochem. Syst. Ecol., 1997, 25: 309-325.
Zheng X.-Q. et al., “Distribution, biosynthesis and function of purine and pyridine alkaloids in Coffea arabica seedlings”, Plant Sci., 2004, 166: 807-813.

1-34. (canceled)

35. A method comprising a step of extracting an aerial part of at least one plant of the Rubiaceae family to obtain an extract comprising at least one C-glycosyl xanthone, wherein the at least one C-glycosyl xanthone is selected from the group consisting of mangiferin, isomangiferin and a combination thereof.

36. The method according to claim 35, wherein the plant of the Rubiaceae family belongs to a subfamily selected from the group consisting of Rubioideae, Cinchonoideae, Ixoroideae, and Antirheoideae.

37. The method according to claim 35, wherein the plant of the Rubiaceae family belongs to a genus selected from the group consisting of Coffea and Rondeletia.

38. The method according to claim 37, wherein the plant belonging to the Coffea genus is selected from the group consisting of Coffea abbayesii, Coffea abeokutae, Coffea affinis, Coffea alleizettii, Coffea ambanjensis, Coffea ambongensis, Coffea andrambovatensis, Coffea ankaranensis, Coffea anthonyi, Coffea arabica L., Coffea arenesiana, Coffea augagneurii, Coffea bakossii, Coffea benghalensis, Coffea bertrandii, Coffea betamponensis, Coffea bissetiae, Coffea boinensis, Coffea boiviniana, Coffea bonnieri, Coffea brevipes, Coffea bridsoniae, Coffea buxifolia, Coffea canephora, Coffea carrissoi, Coffea charrieriana, Coffea commersoniana, Coffea congensis, Coffea costatifructa, Coffea coursiana, Coffea dactylifera, Coffea decaryana, Coffea Dewevrei, Coffea dubardii, Coffea eugenioides, Coffea fadenii, Coffea farafanganensis, Coffea fotsoana, Coffea fragilis, Coffea gallienii, Coffea grevei, Coffea heimii, Coffea heterocalyx, Coffea homollei, Coffea humbertii, Coffea humblotiana, Coffea humilis, Coffea jumellei, Coffea kapakata, Coffea khasiana, Coffea kianjavatensis, Coffea kihansiensis, Coffea kimbozensis, Coffea kivuensis, Coffea Klainii, Coffea labatii, Coffea lancifolis, Coffea leonimontana, Coffea leroyi, Coffea liaudii, Coffea liberica, Coffea ligustroides, Coffea littoralis, Coffea lulandoensis. Coffea macrocarpa, Coffea magnistipula, Coffea mangoroensis, Coffea manombensis, Coffea mapiana, Coffea mauritiana, Coffea mayombensis, Coffea mcphersonii, Coffea millotii, Coffea minutiflora, Coffea mogenetii, Coffea mongensis, Coffea montekupensis, Coffea montis-sacri, Coffea moratii, Coffea mufindiensis, Coffea myrtifolia, Coffea perrieri, Coffea pervilleana, Coffea pocsii, Coffea pseudozanguebariae, Coffea pterocarpa, Coffea quillou, Coffea racemosa, Coffea rakotonasoloi, Coffea ratsimamangae, Coffea resinosa, Coffea rhamnifolia, Coffea richardii, Coffea rupestris, Coffea sahafaryensis, Coffea sakarahae, Coffea salvatrix, Coffea sambavensis, Coffea schliebenii, Coffea sessiliflora, Coffea sp Moloundou, Coffea stenophylla, Coffea tetragona, Coffea togoensis, Coffea travancorensis, Coffea tricalysioides, Coffea tsirananae, Coffea vatovavyensis, Coffea vavateninensis, Coffea vianneyi, Coffea vohemarensis, Coffea wightiana, Coffea zanguebariae, and hybrids thereof.

39. The method according to claim 38, wherein the plant belonging to the Coffea genus is selected from the group consisting of Coffea arabica, Coffea eugenioides, Coffea heterocalyx, Coffea pseudozanguebariae, Coffea sp Moloundou, Coffea Pointed Bourbon, and hybrids thereof.

40. The method according to claim 35, wherein the aerial part of the plant comprises leaves of the plant.

41. The method according to claim 35, wherein the step of extracting is performed using a mixture of water and a polar solvent.

42. The method according to claim 35, wherein the method further comprises grinding the aerial part of the plant prior to extraction.

43. The method according to claim 42, wherein the aerial part of the plant is dried.

44. The method according to claim 35, wherein the step of extracting comprises performing a sonication.

45. The method according to claim 35, wherein the step of extracting comprises using a water/methanol mixture.

46. The method according to claim 35, further comprising a step of isolating at least one C-glycosyl xanthone from the extract by chromatography.

47. The method according to claim 46, wherein isolating at least one C-glycosyl xanthone from the extract comprises performing a medium pressure liquid chromatography on a cellulose column, wherein the cellulose column is eluted using:
water to obtain a first fraction comprising mangiferin, and
then a water/alcohol mixture to obtain a second fraction comprising isomangiferin.

48. The method according to claim 47, further comprising a step of purifying mangiferin from the first fraction using a Sephadex column eluted with water.

49. The method according to claim 47, further comprising a step of purifying isomangiferin from the second fraction using a cellulose column eluted with an alcohol/water mixture.

50. A method according to claim 35, wherein the extracting comprises:
(a) grinding the aerial part of the plant into a powder, wherein the aerial part of the plant consists essentially of young leaves of the plant and the plant belongs to the Coffea genus, and
(b) sonicating the powder at a temperature between about 0° C. and about 5° C. in a water/methanol mixture in a 80:20 (vol:vol) ratio to obtain an extract comprising at least one C-glycosyl xanthine.

51. The method of claim 50, wherein step (a) further comprises drying the powder by lyophilisation.

52. The method of claim 50, further comprising (c) performing one or more of:
(i) isolating at least one C-glycosyl xanthone from the extract by submitting the extract to a medium pressure liquid chromatography on a cellulose column eluted using water to obtain a first fraction comprising mangiferin, and then a water/methanol mixture in a 10:90 (vol:vol) ratio to obtain a second fraction comprising isomangiferin;
(ii) purifying mangiferin from the first extract fraction using a Sephadex column eluted with water; and
(iii) purifying isomangiferin from the second extract fraction using a cellulose column eluted with an ethanol/water mixture in a 80:20 (vol:vol) ratio.

53. An extract comprising at least one C-glycosyl xanthone selected from the group consisting of mangiferin, isomangiferin and a combination thereof, and wherein extract is obtained by a method according to claim 35 or claim 50.

54. The extract according to claim 53, wherein mangiferin is the main component of the extract or isomangiferin is the main component of the extract.

55. A composition comprising an extract according to claim 53 and at least one pharmaceutically acceptable carrier.

56. The composition of claim 55 further comprising at least one pharmaceutical active principle.

57. A composition comprising an extract according to claim 53 and at least one cosmetically acceptable carrier.

58. The composition of claim 57 further comprising at least one cosmetic active principle.
